(19) United States
(12) Patent Application Publication
(10) Pub. No.: US 2017/0328910 A1
(43) Pub. Date: Nov. 16, 2017

(54) PRO-ANGIOGENIC AND ANTI-ANGIOGENIC HEMATOPOIETIC PROGENITOR CELL POPULATIONS

(71) Applicant: Indiana University Research and Technology Corporation, Indianapolis, IN (US)

(72) Inventors: Jamie Case, Indianapolis, IN (US); Julie A. Mund, Indianapolis, IN (US); Myka L. Estes, Davis, CA (US)

(21) Appl. No.: 15/655,417

(22) Filed: Jul. 20, 2017

Related U.S. Application Data
(63) Continuation of application No. 14/207,827, filed on Mar. 13, 2014.
(60) Provisional application No. 61/779,820, filed on Mar. 13, 2013.

(51) Int. Cl. G01N 33/574; G01N 33/50

(52) U.S. Cl. CPC G01N 33/57492; G01N 33/57407; G01N 33/5743; G01N 33/5073; G01N 2333/70596; G01N 2800/7014; G01N 2333/70589

(57) ABSTRACT

Methods for identifying active angiogenesis and vasculopathy are described. More particularly, the present disclosure relates to cellular biomarkers, and to methods of screening cellular biomarkers for identifying active angiogenesis.

### CROSS-REFERENCE TO RELATED APPLICATIONS

This application is a continuation application of U.S. patent application Ser. No. 14/207,827 (published as U.S. Publication No. 2014/0273017) filed Mar. 13, 2014, which claims priority to U.S. Provisional Patent Application No. 61/779,820 filed on Mar. 13, 2013, both of which are hereby incorporated by reference in their entireties.

### BACKGROUND OF THE DISCLOSURE

The present disclosure relates generally to biomarkers and their use for predicting active angiogenesis. More particularly, the present disclosure relates to cellular biomarkers, and to methods of screening cellular biomarkers for identifying active angiogenesis.

The tumor microenvironment contains various cell subpopulations of hematopoietic and endothelial origin. These cells are recruited by the tumor and play an integral role in regulating tumor growth and metastasis. However, the heterogeneity of these cells has made it difficult to study their specific function in tumorigenesis.

Angiogenesis, the formation of new blood vessels from pre-existing vessels, and neovascularization (neoangiogenesis) are recognized as having an important role in tumor growth. Tumors induce angiogenesis by secreting various growth factors such as vascular endothelial growth factor (VEGF). Anti-angiogenic therapies are being tested for slowing or preventing growth of tumors. For example, increased survival rates have been observed with the addition of antibodies specific for VEGF as compared to contemporary therapy for colon and rectal cancers.

Vasculopathy is also associated with a variety of diseases and disorders. One group in particular that is affected includes subjects who have undergone or who are undergoing cancer treatment. Many cancer treatments have been associated with medical conditions such as cardiovascular and cerebrovascular side-effects. Vascular endothelial damage due to chemo-radiotherapy has emerged as a significant cause of cardiovascular disease in subjects who have undergone or who are undergoing cancer treatment.

Although screening tests for angiogenesis and vasculopathy have been recommended, there are no established peripheral blood (PB) biomarkers for identifying active angiogenesis. Accordingly, there exists a need to develop screening methods for identifying active angiogenesis.

### BRIEF DESCRIPTION OF THE DISCLOSURE

The present disclosure is generally directed to methods for screening for peripheral blood and bone marrow cellular biomarkers for identifying angiogenesis. More particularly, the present disclosure is directed to methods for screening peripheral blood and bone marrow for a ratio between pro-angiogenic circulating hematopoietic stem and progenitor cells (pCHSPC) to anti-angiogenic circulating hematopoietic stem and progenitor cells (aCHSPC) to identify active angiogenesis.

In one aspect, the present disclosure is directed to a method for identifying active angiogenesis in a subject having or suspected of having cancer. The method comprises: obtaining a peripheral blood sample from the subject; determining a ratio of pro-angiogenic circulating hematopoietic stem and progenitor cells (pCHSPC) to anti-angiogenic circulating hematopoietic stem and progenitor cells (aCHSPC) for the peripheral blood sample; and identifying active angiogenesis in the subject if the ratio is greater than 2.0.

In another aspect, the present disclosure is further directed to a method for identifying active angiogenesis in a subject having or suspected of having cancer. The method comprises: obtaining a bone marrow sample from the subject; determining a ratio of pro-angiogenic circulating hematopoietic stem and progenitor cells (pCHSPC) to anti-angiogenic circulating hematopoietic stem and progenitor cells (aCHSPC) for the bone marrow sample; and identifying active angiogenesis in the subject if the ratio is greater than 2.0.

In another aspect, the present disclosure is also directed to a method of screening cellular biomarkers in a peripheral blood sample for identifying active angiogenesis in a subject having or suspected of having cancer. The method comprises: obtaining a peripheral blood sample from the subject; determining by multi-parametric flow cytometry a number of pro-angiogenic circulating hematopoietic stem and progenitor cells (pCHSPC) in the peripheral blood sample; determining by multi-parametric flow cytometry a number of anti-angiogenic circulating hematopoietic stem and progenitor cells (aCHSPC) in the peripheral blood sample; and calculating the ratio of pro-angiogenic circulating hematopoietic stem and progenitor cells (pCHSPC) to anti-angiogenic circulating hematopoietic stem and progenitor cells (aCHSPC); and identifying active angiogenesis in the subject if the ratio is greater than 1.8.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic of a C32 Melanoma Xenograft Model. Mice were sub-lethally irradiated with 300 rads and human CD34⁺ cells were then transplanted. Following 4 weeks of engraftment, peripheral blood was analyzed for human CD45 and then C32 melanoma cells were implanted on the flanks of the humanized mice with tumor growth and levels of CHSPCs both being monitored.

FIGS. 2A-2C depict the comparison of proangiogenic circulating hematopoietic stem/progenitor cells (pCHSPCs) as discussed in Example 1. PCHSPCs (AC133⁺) were mobilized from the bone marrow into the peripheral blood following 4 weeks after C32 melanoma flank implantation (p=<0.005).

FIG. 3 depicts the increased tumor volume following CD34⁺ transplant as discussed in Example 1. Tumor volume was increased in animals that underwent a CD34⁺ cell transplantation after sub-lethal irradiation compared to those injected with saline and were then implanted with C32 melanoma in a flank model (p=<0.01).

FIG. 4 depicts the decreased survival in an orthotopic glioblastoma model following proangiogenic circulating hematopoietic stem/progenitor cell (pCHSPC) injection as discussed in Example 1. Mice were implanted with GBM10 glioblastoma in an intracranial model and were injected with one of: a vehicle, pCHSPCs at 15 days, or pCHSPCs at 21 days post implant. Mice injected with pCHSPCs had a decreased survival time compared to vehicle controls.

FIGS. 5A-5C depict the treatment of C32 implanted mice with interferon decreased tumor fold change as discussed in Example 1. Mice were first humanized using a CD34⁺ xenograft and C32 melanoma was implanted on the flank. Following two weeks of C32 melanoma unrestricted growth, some mice were treated with Interferon alpha-2b (Intron-A; 50,000 U×3 times a day) and tumor fold change was measured. Mice receiving Intron-A had a decreased tumor fold change compared to untreated animals (p=<0.005).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, “pro-angiogenic circulating hematopoietic stem and progenitor cell (pCHSPC)” refers to circulating hematopoietic stem and progenitor cells with the following surface antigen expression:

CD45^{dim}CD34⁺CD31⁺AC133⁺CXCR4⁺CD14⁻CD16⁻LIVE/DEAD⁻CD41a⁻.

As used herein, “anti-angiogenic circulating hematopoietic stem and progenitor cell (aCHSPC)” refers to circulating hematopoietic stem and progenitor cells with the following surface antigen expression: CD45^{dim}CD34⁺CD31⁺AC133⁻CD14⁻CD16⁻LIVE/DEAD⁻CD41a⁻.

As used herein, a “normal, healthy individual” or “normal, healthy subject” refers to a subject that does not have the specific disease, disorder or condition being tested. For example, in one embodiment, a normal, healthy subject being tested for active angiogenesis or vasculopathy has a pCHSPC:aCHSPC ratio of from about 1.2 to about 1.8.

As used herein, “a subject in need thereof” refers to a subject susceptible to or at risk of a specified disease, disorder, or condition. More particularly, in the present disclosure the methods of screening cellular biomarkers in bone marrow can be used with a subset of subjects who are susceptible to or at elevated risk for experiencing vasculopathy as a side-effect of cancer treatment. Such subjects may include, but are not limited to, subjects susceptible to or at elevated risk of various forms of cancer such as, for example, acute lymphoblastic leukemia, non-Hodgkin's lymphoma, central nervous system-primitive neuro-ectodermal tumor, and central nervous system germ cell tumor. Subjects may be susceptible to or at elevated risk for cancer due to family history, age, environment, and/or lifestyle. In other embodiments, subjects who may be susceptible to or at elevated risk for experiencing vasculopathy may include, but are not limited to, subjects susceptible to or at elevated risk of obesity, diabetes, hyperlipidemia, hypertension, chronic kidney disease, hypercholesterolaemia, atherosclerosis, cardiovascular disease, cerebrovascular complications, peripheral vascular disease, congestive heart failure, ischemic heart disease, vascular inflammation, or sickle cell disease. Subjects may be susceptible to or at elevated risk for the above-named diseases, disorders, and conditions due to family history, age, environment, and/or lifestyle.

In yet other embodiments, subjects may be susceptible to or at elevated risk for experiencing other conditions such as active angiogenesis. Subjects who may be susceptible to, or at elevated risk for experiencing active angiogenesis may include, but are not limited to, subjects susceptible to or at elevated risk of cancer, undergoing cancer treatment, or pregnant subjects. Subjects may be susceptible to or at elevated risk for cancer, cancer treatment, or pregnancy due to family history, age, environment, and/or lifestyle.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

As used herein, “susceptible” and “at risk” refer to having little resistance to a certain disease, disorder or condition, including being genetically predisposed, having a family history of, and/or having symptoms of the disease, disorder or condition.

Generally, the methods of the present disclosure have identified circulating hematopoietic stem and progenitor cells (CHSPC) having pro-angiogenic properties as being potential cellular biomarkers for identifying subjects with active angiogenesis and vasculopathy. Particularly, it has been found that the ratio of pro-angiogenic circulating hematopoietic stem and progenitor cells (pCHSPCs) to anti-angiogenic circulating hematopoietic stem and progenitor cells (aCHSPCs) can indicate if a subject has or is at risk for active angiogenesis, vasculopathy, or diseases and conditions relating thereto (e.g., cancer, undergoing anti-cancer treatment/therapy (e.g., chemotherapy, antibody therapy, radiation therapy and combinations thereof, and the like), pregnancy, diabetes, sickle cell disease, vascular inflammation, and the like).

In one aspect, the present disclosure is directed to a method of screening cellular biomarkers in peripheral blood for identifying active angiogenesis in a subject. The method includes obtaining a peripheral blood sample from the subject; determining a ratio of pro-angiogenic circulating hematopoietic stem and progenitor cells (pCHSPC) to anti-angiogenic circulating hematopoietic stem and progenitor cells (aCHSPC) for the peripheral blood sample; and identifying active angiogenesis in the subject if the ratio is greater than 2.0.

In another aspect, the present disclosure is directed to a method of screening cellular biomarkers in a bone marrow sample for identifying active angiogenesis in a subject. The method includes: obtaining a bone marrow sample from the subject; determining a ratio of pro-angiogenic circulating hematopoietic stem and progenitor cells (pCHSPC) to anti-angiogenic circulating hematopoietic stem and progenitor cells (aCHSPC) for the bone marrow sample; and identifying active angiogenesis in the subject if the ratio is greater than 2.0.

In yet another aspect, the present disclosure is directed to a method of screening cellular biomarkers in a peripheral blood sample for identifying active angiogenesis in a subject having or suspected of having cancer. The method includes: obtaining a peripheral blood sample from the subject; determining by multi-parametric flow cytometry a number of pro-angiogenic circulating hematopoietic stem and progenitor cells (pCHSPC) in the peripheral blood sample; determining by multi-parametric flow cytometry a number of anti-angiogenic circulating hematopoietic stem and progenitor cells (aCHSPC) in the peripheral blood sample; and calculating the ratio of pro-angiogenic circulating hematopoietic stem and progenitor cells (pCHSPC) to anti-angiogenic circulating hematopoietic stem and progenitor cells (aCHSPC); and identifying active angiogenesis in the subject if the ratio is greater than 1.8.

As used herein “angiogenesis” refers to the expansion of current blood vessels or the creation of new blood vessels. “Physiological angiogenesis” is coordinated by complex molecular and cellular mechanisms and is necessary for optimum vascular endothelial health. As used herein “active angiogenesis” refers to the expansion of current blood vessels or the creation of new blood vessels above normal/regular vascular repair; that is vascular repair in a subject not having angiogenesis. The method includes screening peripheral blood from a subject for a ratio between pro-angiogenic circulating hematopoietic stem and progenitor cell (pCHSPC) to anti-angiogenic circulating hematopoietic stem and progenitor cell (aCHSPC) and identifying active angiogenesis in the subject if the ratio is greater than 2.0. To determine the ratio of pCHSPC to aCHSPC, peripheral blood can be screened using multi-parametric flow cytometry. Peripheral blood is collected from a subject using any methods known in the art for blood collection.

In one embodiment, the subject has cancer. In another embodiment, the subject is at risk for having cancer. In another embodiment, the subject is diagnosed with cancer. The cancer can be, for example, melanoma and glioblastoma.

The cells are stained with antibodies and acquired or sorted using flow cytometry. Antibodies for identifying and distinguishing pCHSPC and aCHSPC include antibodies that specifically bind to cell surface molecules including: CD45, CD34, CD31, AC133, CD14, CD16, LIVE/DEAD, CD41a, CXCR4 and CD38.

For the system to perform multi-parametric flow cytometry, digital equipment is preferred over analog equipment for binning fluorescence. Digital equipment has 200,000+ channels for binning the fluorescence compared to 2,200 channels in previously used analog equipment. This allows more sensitive detection between cells of similar apparent brightness. Fluorescent minus one (FMO) gating controls in which cells are stained with all of the reagents except the reagent for which the positive threshold is determined, can be used to determine the threshold separating negative populations from dully fluorescent cells. Single-color compensation controls can be used to calculate the compensation correction value for each fluorochrome. To generate single-color compensation controls, each of the fluorochromes can be used to stain compensation beads (such as, for example, Ig BD CompBeads commercially available from BD Biosciences, San Jose, Calif.) and mononuclear cells. The use of single stained bead controls with titrated antibodies specific to each lot of each marker allows for creating a compensation matrix in the analysis software that is accurate for all samples for a given run. Exporting files such as, for example, FCS 3.0, having bi-exponential display allows seeing above and below the axis. Fluorescence is relative; there are no fixed values. This shows the values in the correct expressions on the y- and x-axis. Acquiring the data uncompensated removes the bias of compensation by eye that can lead to false positives.

It has been found that a pCHSPC:aCHSPC ratio in a subject of greater than 1.8, preferably greater than 2.0, can indicate active angiogenesis in the subject such as a subject who has cancer, a subject who is diagnosed with cancer, a subject who is undergoing treatment for cancer, or combinations thereof. Further, in some embodiments, a ratio of pCHSPC:aCHSPC greater than 2.0 can indicate active angiogenesis associated with pregnancy.

The method can further include administering anti-angiogenic circulating hematopoietic stem and progenitor cell (aCHSPC) to a subject in need thereof. Administration of aCHSPC may reduce/minimize/inhibit active angiogenesis in the subject. Accordingly, administering aCHSPC to a subject, may reduce/minimize/inhibit symptoms or the progression of diseases, disorders, or conditions associated with active angiogenesis. Subjects who may benefit from such administration include subjects who may be susceptible to, or at elevated risk for experiencing active angiogenesis associated with cancer, undergoing cancer treatment, or pregnant subjects.

In another aspect, the present disclosure is directed to a method of screening cellular biomarkers in peripheral blood for identifying vasculopathy in a subject. It has been found that a pCHSPC:aCHSPC ratio in a subject of less than 1.0 can indicate vasculopathy in the subject. As used herein, “vasculopathy” refers to diseases and disorders of the blood vessels. Diseases and conditions associated with or that lead to vasculopathy can be, for example, cancer, in particular, subjects subjected to cancer treatment, obesity, diabetes, hyperlipidemia, hypertension, chronic kidney disease, hypercholesterolaemia, atherosclerosis, cardiovascular disease, cerebrovascular complications, peripheral vascular disease, congestive heart failure, ischemic heart disease, sickle cell disease, and vascular inflammation.

The method can further include administering pro-angiogenic circulating hematopoietic stem and progenitor cells (pCHSPC) to a subject with or at risk of vasculopathy. Administration of pCHSPC may reduce/minimize/inhibit vasculopathy or the risk thereof in the subject in need thereof. Accordingly, administering pCHSPC to a subject in need thereof, may reduce/minimize/inhibit symptoms or the progression of diseases, disorders, or conditions associated with vasculopathy. Subjects who may benefit from such administration include subjects who may be susceptible to, or at elevated risk for experiencing vasculopathy associated with cancer, cancer treatment, obesity, diabetes, hyperlipidemia, hypertension, chronic kidney disease, hypercholesterolaemia, atherosclerosis, cardiovascular disease, cerebrovascular complications, peripheral vascular disease, congestive heart failure, ischemic heart disease, vascular inflammation, and sickle cell disease.

The method can further include administering a treatment to inhibit or mitigate the development of vasculopathy. As used herein, “mitigate” refers to slowing/eliminating the development or progression of a disease, disorder or condition and/or reducing/eliminating the symptoms of a disease, disorder or condition. Suitable treatments can be, for example, modification of lifestyle (e.g., diet and exercise), modification of diet, administration of medications to lower blood pressure, administration of medications to lower cholesterol and lipids (e.g., statins, fibrates, ezetimibe, colesevelam, torcetrapib, avasimibe, implitapide, and niacin), and combinations thereof.

In addition to the methods described above, it has now been surprisingly recognized that bone marrow can also be screened using the methods of the present disclosure to identify subjects having and/or at risk of developing vasculopathy and its related diseases, disorders and conditions. For example, in one embodiment, the present disclosure is directed to a method of screening cellular biomarkers in bone marrow for identifying a risk for vasculopathy as a side-effect of cancer treatment for a subject in need thereof. The method includes obtaining a bone marrow sample from the subject; and determining a ratio of pCHSPC to aCHSPC for the bone marrow sample. A pCHSPC to aCHSPC ratio that is less than 1.0 is indicative that the subject is at risk for developing vasculopathy as a side-effect of cancer treatment.

The cancer treatment can be, for example, a chemotherapy, antibody therapy, radiation therapy and combinations thereof.

In another aspect, using the methods of the present disclosure can be used to screen cellular biomarkers in peripheral blood for identifying vasculopathy as a side-effect of cancer treatment. Following cancer treatment, subjects commonly experience artherosclerotic cardiovascular and cerebrovascular complications such as, for example, stroke and aneurysm formation/rupture, as a result of treatment-induced late-effects.

The method can further include administering a treatment to inhibit or mitigate the development of cardiovascular disease or cerebrovascular complications in subjects who have undergone or who are undergoing cancer treatment. As used herein, “mitigate” refers to slowing/eliminating the development or progression of a disease, disorder or condition and/or reducing/eliminating the symptoms of a disease, disorder or condition. Suitable treatments can be, for example, modification of lifestyle (e.g., diet and exercise), modification of diet, administration of medications to lower blood pressure, administration of medications to lower cholesterol and lipids (e.g., statins, fibrates, ezetimibe, colesevelam, torcetrapib, avasimibe, implitapide, and niacin), and combinations thereof.

The method can further include administering pro-angiogenic circulating hematopoietic stem and progenitor cell (pCHSPC) to a subject having or at risk for cardiovascular disease or cerebrovascular complications as a result of cancer treatment. Administration of pCHSPC may reduce/minimize/inhibit cardiovascular disease or cerebrovascular compositions or the risk thereof in the subject.

In another aspect, the present disclosure is directed to a method for monitoring the efficacy of an anti-cancer treatment. The method includes obtaining a peripheral blood sample from a subject prior to undergoing anti-cancer treatment; determining a pre-treatment ratio of pCHSPC to aCHSPC for the peripheral blood sample; administering the anti-cancer treatment; obtaining a post-treatment peripheral blood sample from the subject; and determining a post-treatment ratio of pCHSPC to aCHSPC for the post-treatment peripheral blood sample, wherein a lower post-treatment ratio of pCHSPC to aCHSPC as compared to the pre-treatment ratio of pCHSPC to aCHSPC indicates effectiveness of the treatment.

The anti-cancer treatment can be, for example, chemotherapy, antibody therapy, radiation therapy, and combinations thereof. The anti-cancer treatment can be administered for a period of about 21 days.

In one embodiment, the pre-treatment ratio of pCHSPC to aCHSPC is greater than 2.0. The post-treatment ratio of pCHSPC to aCHSPC ranges from 1.2 to 1.8.

In another aspect, the present disclosure is directed to a method of preparing a murine xenograft model for cancer. The method includes irradiating a mouse with a dose of from about 250 cGy to about 350 cGy total body irradiation using methods known to those skilled in the art. Suitable mice strains can be, for example, a NOD. Cg-Prkdc^{scid}IL2rg^{tmlWjl}/Sz (NOD/SCID/γchain^{null}) mouse.

Human cells to be transplanted into the mouse can be, for example, CD34⁺ cells, pCHSPCs, aCHSPCs, and combinations thereof. CD34⁺ cells, pCHSPCs, aCHSPCs, and combinations of these cells can be isolated from humans using methods known to those skilled in the art. In particular, Magnetic Cell Sorting (MACS) System can be used to isolate human CD34⁺ cells. Once isolated, the human cells are transplanted into the mouse. Suitable amounts of cells to be transplanted can be from about 5×10⁴ cells per mouse to about 2×10⁵ cells per mouse, including about 10⁵ cells per mouse.

Any source for isolating the human CD34⁺ cells, pCHSPCs and aCHSPCs can be used. Human umbilical cord blood is a particularly suitable source for isolating the human CD34⁺ cells, pCHSPCs and aCHSPCs. The human CD34⁺ cells (including whole CD34⁺ cells), pCHSPCs and aCHSPCs can be transplanted to the mouse using any method known by those skilled in the art. Transplantation by tail vein injection is a particularly suitable method for transplanting cells.

The method can further include determining CD34⁺ cells, pCHSPCs and aCHSPCs in peripheral blood after transplantation. Suitable methods for determining CD34⁺ cells, pCHSPCs and aCHSPCs in peripheral blood can be, for example, flow cytometry. A particularly suitable method for determining CD34⁺ cells, pCHSPCs and aCHSPCs in peripheral blood can be, for example, multi-parametric flow cytometry as described herein. CD34⁺ cells, pCHSPCs and aCHSPCs in peripheral blood can be determined over a time period of from about 1 week to about 4 weeks after implantation, for example. Longer time periods can be used to monitor CHSPC release from bone marrow into peripheral blood.

Human cancer cells are then injected into the mouse. Suitable human cancer cells that can be injected into the mouse can be, for example, C32 cells and glioblastoma cells. Suitable amounts of cancer cells to be injected can be from about 5×10⁵ cells per mouse to about 4×10⁶ cells per mouse, including about 2×10⁶ cells per mouse.

In accordance with the present disclosure, methods have been discovered that surprisingly allow for the screening of peripheral blood for the identification of active angiogenesis and vasculopathy. Methods have also been discovered that allow for the screening of cellular biomarkers in bone marrow for the identification of vasculopathy. Advantageously, the methods of the present disclosure provide cellular biomarkers to identify active angiogenesis and vasculopathy, which can be used to monitor response to angiogenesis-promoting and anti-angiogenesis treatments, as well as to identify and/or monitor vasculopathy as a side-effect of medical treatments for diseases that affect the cardiovascular system and to incorporate primary preventative and/or early treatment therapies to inhibit or mitigate vasculopathy.

The disclosure will be more fully understood upon consideration of the following non-limiting Examples.

### EXAMPLES

### Example 1

In this Example, two murine orthotopic cancer models utilizing the human cellular biomarker were used to analyze the progression of tumor growth.

### Materials and Methods

Isolation of Umbilical Cord Blood CD34⁺Cells. Samples of human umbilical cord blood (UCB) were collected from normal, full term infants delivered by cesarean section and the CD34⁺ cells were selected using the human CD34 indirect MicroBead kit and Magnetic Cell Sorting (MACS) system (Miltenyi Biotec, Auburn, Calif.) as directed by the manufacturer. The CD34⁺ fraction was subsequently isolated, with the viability of the CD34⁺ cells always greater than 95%. The purity and functionality of the MACS isolated CD34⁺ cells was confirmed by flow cytometry analysis (>95%) and a colony forming unit-granulocyte, erythrocyte, monocyte, megakaryocyte (CFU-GEMM) assay.

Colony Forming Unit Assay. CFU assays (MethoCult GF H4434, Stem Cell Technologies, Inc., Vancouver, Canada) were conducted using the MACS isolated CD34⁺ cells, or the subsets of the CHPSCs. The cells were seeded in 35 mm dishes in triplicate at a concentration of 0.5×10³ CD34⁺ cells per plate in order to obtain approximately 60-70 colonies per dish.

Antibodies and Staining Reagents. The following primary conjugated monoclonal antibodies were used: anti-human CD31 fluoroscein isothyocyanate (FITC, BD PHARMINGEN, Taguig City, Philippines), anti-human CD34 phycoerythrin (PE, BD PHARMINGEN, Taguig City, Philippines), anti-human CD38 phycoerythrin (PE, BD PHARMINGEN, Taguig City, Philippines), anti-human AC133 allophycocyanin (APC, Miltenyi Biotec, Auburn, Calif.)), anti-human CXCR4 allophycocyanin (APC, BD PHARMINGEN, Taguig City, Philippines), anti-human CD16 phycoerythrin-Cy7 (PECy7, BD PHARMINGEN, Taguig City, Philippines), anti-human CD14 PECy5.5 (Invitrogen, Grand Island, N.Y.), anti-human CD45 APC-AlexaFluor (AF) 750 (Invitrogen, Grand Island, N.Y.), anti-human CD235a (glyA, R&D Systems, Minneapolis, Minn.) conjugated to Pacific Blue (PacB, Invitrogen, Grand Island, N.Y.) and the amine reactive viability dye, LiveDead (Invitrogen, Grand Island, N.Y.).

To resolve the rare and/or dim populations of interest, specific antigen and fluorochrome conjugate coupling was optimized for the nine-antibody, plus viability marker staining panel described below.

Multi-Parametric Flow Cytometry Immunostaining and Sorting. MACS isolated CD34⁺ cells were incubated with Fc blocking reagent (Miltenyi Biotec, Auburn, Calif.) and stained as described in Estes et al., Application of polychromatic flow cytometry to identify novel subsets of circulating cells with angiogenic potential. Cytometry Part A: The Journal of the International Society for Analytical Cytology, 2010, September; 77(9): 831-839; and Estes et al., Identification of endothelial cells and progenitor cell subsets in human peripheral blood. Current Protocols in Cytometry. 2010 April; Chapter 9: unit 9 (33): 1-11. “Fluorescent minus one” (FMO) gating controls were also used to ensure proper gating. Briefly, cells were incubated with antibodies for 30 minutes at 4° C., washed twice in PBS with 2% fetal bovine serum (FBS), and were run fresh on a BD Aria Flow cytometer (BD, Franklin Lakes, Franklin Lakes, N.J.) equipped with a 405 nm violet laser, 488 nm blue laser and 633 nm red laser. Data was acquired compensated using anti-mouse Ig BD CompBeads (BD Biosciences, San Jose, Calif.) stained with each of the individual test antibodies to serve as single-color compensation controls. Acquisition files were exported as FCS 3.0 files and analyzed using FlowJo software, version 8.7.3 (Tree Star, Inc., Ashland, Oreg.). Prior to use, each lot of antibody was individually titered as described in Herzenberg et al., Interpreting flow cytometry data: a guide for the perplexed. Nat Immunol. 2006 July; 7(7): 681-685 to determine the optimal staining concentration.

Mice. NOD.CB17-Prkdc^{scid}/j (NOD/SCID), NOD. Cg-Prkdc^{scid}IL2rg^{tmlWjl}/Sz (NOD/SCID/γchain^{null}), or SCID/beige (SCID^{bg}) mice, 6-8 weeks old, were housed according to protocols approved by the Indiana University Laboratory Animal Research Center and adhered strictly to National Institutes of Health guidelines.

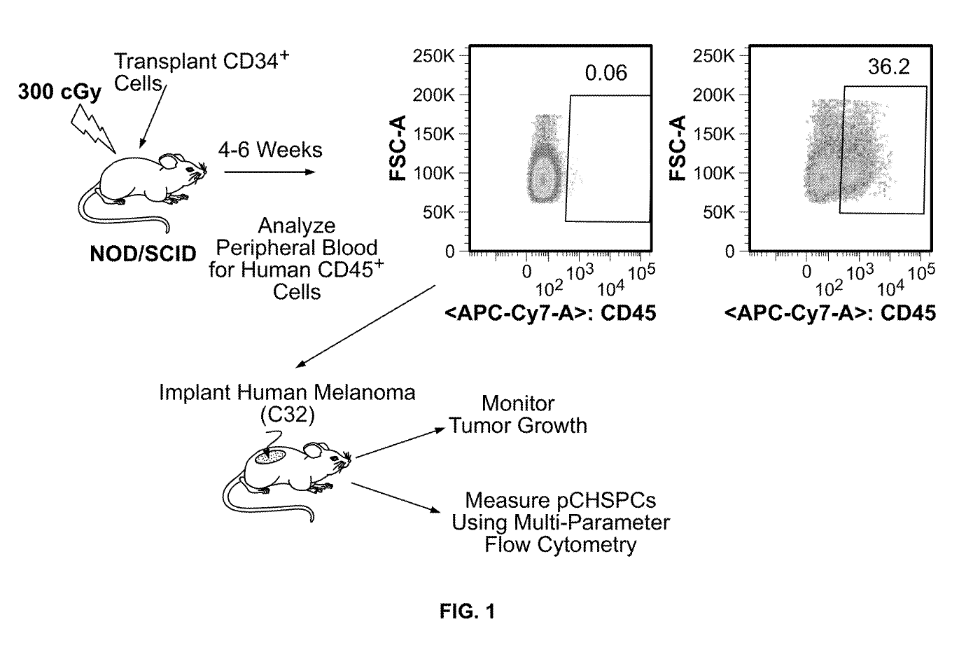

Transplantation of NOD.CB17-Prkdc^{scid}/j, NOD/SCID/γchain^{null} or SCID^{bg} Mice. Generally, the transplantation method is shown in FIG. 1. All animals were given a sub-lethal dose of 300 cGy total body irradiation 4 hours before transplantation. The MACS isolated CD34⁺ cells (10⁵ per mouse) were re-suspended in Dulbecco's Modified Eagle Medium (DMEM, Gibco, Invitrogen, Grand Island, N.Y.) and transplanted by tail vein injection. To assess human engraftment, mice were bled at 4 weeks post transplantation and the PB cells collected using a red blood cell lysis and stained with the human antibodies listed above. Approximately 150,000 events per sample were collected on a BD LSRII flow cytometer. Data was run uncompensated and exported as FCS 3.0 files, with analysis performed utilizing FlowJo software version 8.7.3.

Melanoma and Glioblastoma Xenograft Models. (NOD/SCID), NOD/SCID/γchain^{null}, or SCID^{bg} mice were subcutaneously injected with 2×10⁶ C32 human melanoma cells (ATCC CRL-1585) and tumor growth monitored over time. Tumor growth was monitored by caliper, and the volume determined using the formula: mm³=(width)²×length×0.5. The fold increase in tumor growth was determined by comparing tumor volume over time to that of the base line tumor volume. In some experiments, PB was collected at 1 week, 2 weeks and 4 weeks after implantation so as to monitor the CHSPC release from the bone marrow into the PB using flow cytometry. Interferon alpha-2b (Intron A; 50,000 U×3 times a week) was given via subcutaneous injection and at rotating sites to avoid irritation of the injection sites, for 6 total doses. Intracranial implants were performed as previously described in Sarkaria et al. (Clin. Cancer Res. 2006, 12(7 Pt. 1):2264-2271) and Giannini et al. (Neuro-oncology 2005; 7(2):164-76) using a digitalized stereotaxic delivery system (David Kopf Instruments, Model 5000 microinjection unit, Tujunga, Calif.). For stereotaxic delivery of tumor cells, mice were placed under general anesthesia (ip injection of 16 mg/kg xylazine and 150 mg/kg ketamine) and a digitalized drill assembly was used to bore a hole 0.3 mm in depth and 0.8 mm diameter in the cranium at a position 0.5 mm anterior and 1.2 mm lateral to the bregmal anatomical landmark. Tumor cells (1×10⁶ in 10 μl of RPMI medium) were slowly introduced using a 10 μl Hamilton syringe at a depth of 3.5 mm at a rate of 2 μl/min. Once injection was complete, the needle was kept in place for at least 5 minutes and then slowly removed. The hole was sealed with bone wax and the incision was closed with vetbond. For survival experiments, a pre-death endpoint scoring system was used that was based on body weight, grooming, activity level, and degree of paralysis. Mice were monitored daily and euthanized when moribund.

At the end of the experiment, mice were euthanized, tumors, PB and bone marrow were harvested, and the weight of each tumor was determined. Data are presented as the mean±sem.

Statistical Analysis. Statistical analysis was performed using GraphPad Prism software, version 5.01 for Windows (GraphPad Software, San Diego, Calif.). Data was tested for normality using the D'Agostino-Pearson normality test (alpha=0.05), and normal data sets were compared using two-tailed Student's t test or one-way ANOVA.

### Results

Humanized Bone Marrow and Orthotopic Models Allowed the Monitoring of Mobilization of pCHSPCs in Response to Tumor Growth. It was hypothesized that pCHSPCs could be mobilized from the bone marrow to the PB, which would lead to increased tumor growth. NOD/SCID, NOD/SCID/γchain^{null}, or SCID^{bg} mice were sub-lethally irradiated and UCB MACS isolated CD34⁺ cells or pCHSPC fraction was injected via the tail vein to compare the engraftment potential of all 3 strains. pCHSPCs were first separated using a CD34⁺ magnetic separation and then stained and sorted utilizing a BD Aria to ensure their phenotype. Engraftment was checked at 4 weeks post transplantation by quantifying the number of human CD45⁺ cells in the PB. NOD/SCID and NOD/SCID/γchain^{null} mice had similar engraftment whether whole CD34⁺ cells or the pCHSPC fraction was used (data not shown). Interestingly, SCID^{bg} mice failed to engraft when either whole CD34⁺ cells or the pCHSPC fraction was injected (data not shown).

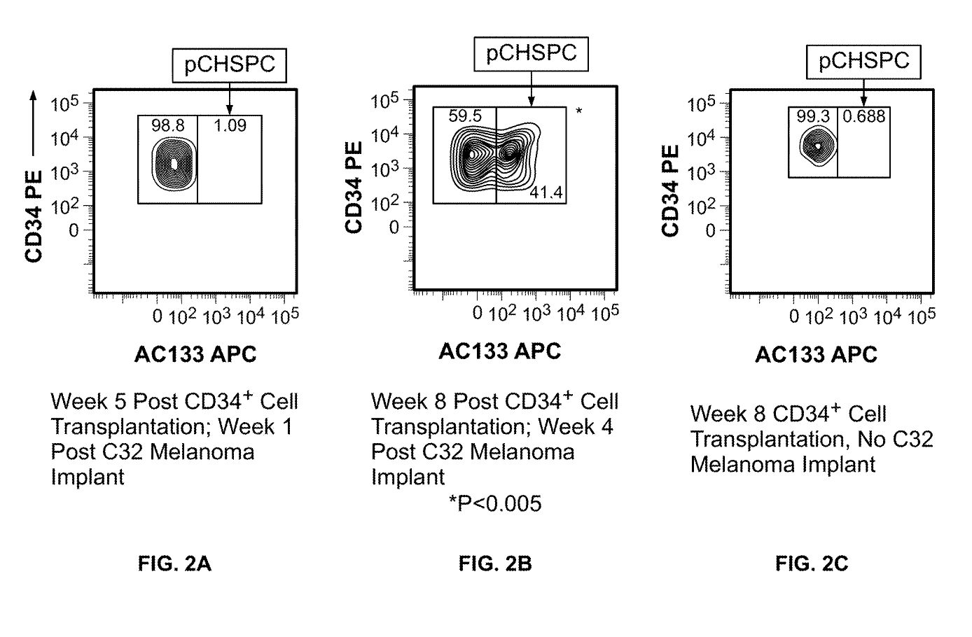

After determining that the pCHSPCs could repopulate the bone marrow niche of both NOD.CB17-Prkdc^{scid}/j and NOD/SCID/γchain^{null} mice, PB was collected to determine whether human pCHSPCs would be detected using multi-parametric flow cytometry as a way to monitor active angiogenesis in response to tumor burden. PB in humanized mice had detectable levels of the parent population of the CHSPCs, with very few pro-angiogenic cells (pCHSPCs) (FIG. 2A). Following flank implantation of C32 melanoma cells, PB was drawn each week and CHSPCs were quantified. While the tumors grew at a slow rate over weeks 1-2, the number of pCHSPCs remained low. Following four weeks post implantation, when the tumors started to rapidly grow, there was a statistically significant increase in the number of pCHPSCs (FIG. 2B; p=<0.005) as compared to control animals (no C32 Transplantation Melanoma Implant; see, FIG. 2C). Interestingly, there was a mobilization of the pCHSPC fraction while the aCHSPC fraction remained at a stable level. This subsequently increased the pCHSPC:aCHSPC ratio.

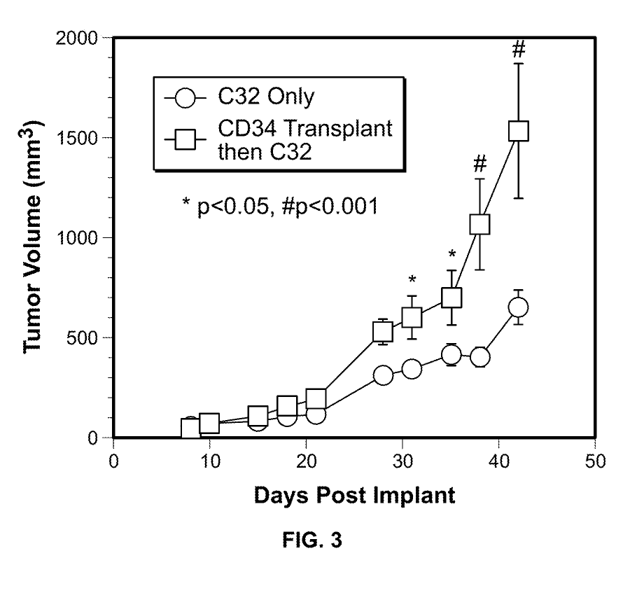

To assess whether this increase in pCHSPCs was responsible for increased tumor size, mice were implanted with C32 melanoma following sub-lethal irradiation and implantation of either CD34⁺ cells or saline. Following 4 weeks of recovery and engraftment, C32 melanoma was implanted on the flank of mice and tumor volumes were measured for 45 days (FIG. 3). Tumors in both C32 only mice and CD34⁺ transplanted mice grew at similar rates until about 25 days post implant. After 25 days, the CD34⁺ transplanted mice began to increase tumor volume more rapidly, which corresponded with the increase in detectable pCHSPCs in the PB (FIG. 2B; FIG. 3). At 45 days post tumor implant, the C32 only cohort had statistically significantly smaller tumor volumes compared to those mice that received CD34⁺ cells following irradiation and subsequent C32 implantation (FIG. 3; p=<0.01).

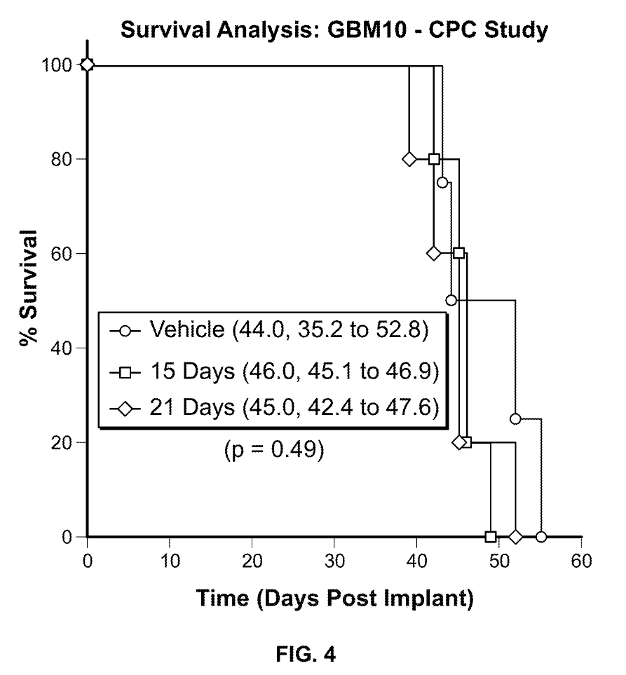

Decreased Survival Following Injection of pCHSPCs in an Orthotopic Glioblastoma Model. Using an orthotopic model of glioblastoma in NSG mice, pCHSPCs were injected at either Day 15 or Day 21 post tumor implant; saline was used as a control. Mice injected with pCHSPCs had decreased survival compared to saline (FIG. 4). Though not statistically significant (due to the small numbers used, n=0), this trend indicates that pCHSPCs play a role in the survival and increased progression of tumors to lethal endpoints.

Quantification of the pCHSPCs in a Humanized Bone Marrow Xenograft can be used as a Biomarker for Evaluation of Anticancer Therapies in Orthotopic Models of Cancer. As the monitoring of pCHSPCs can be performed in a humanized mouse model in response to two orthotopic cancer models, it was then hypothesized that treatment of the mice with anticancer therapy could decrease the number of pCHSPCs, and thus the tumor. NOD/SCID/γchain^{null} mice were irradiated and CD34⁺ cells were injected via tail vein and the mice were left to reconstitute their bone marrow for 4 weeks. Following baseline bleeds for human CD45⁺ and pCHPSC:aCHSPC measurements, C32 cells were implanted in the flank of the animals.

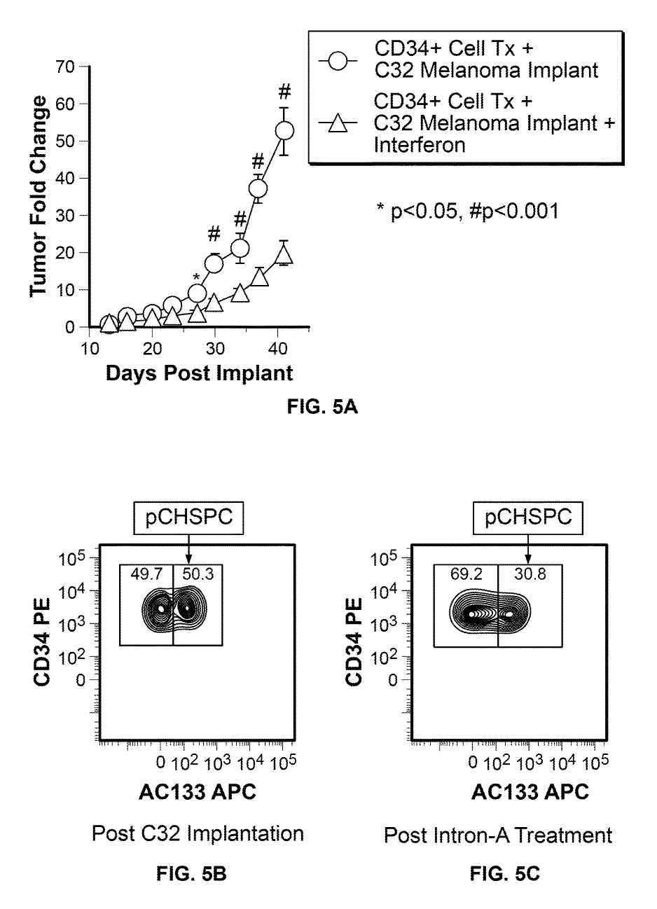

Two weeks later, treatment with the chemotherapeutic agent Interferon alpha-2b (Intron A; 50,000 U×3 times a week) was started and tumor size was measured for 2 weeks post treatment. Following treatment with Interferon for two weeks, at time of harvest, the tumor fold change was statistically significantly decreased compared to the untreated tumor bearing mice (p=<0.005; FIGS. 5A-5C). In addition, the pCHSPC:aCHSPC ratio was significantly decreased in the same mice from two weeks post implantation of C32 cells to 2 weeks of Interferon treatment (p=<0.05) (FIGS. 5A-5C).

### Discussion

The ability to track a human cellular biomarker of angiogenesis was demonstrated during the progression of tumor growth in two murine orthotopic cancer models. Tracking of the pCHSPCs in the PB of both tumor bearing and non-tumor bearing humanized mice demonstrated its potential as a useful biomarker of angiogenesis within in vivo cancer and anticancer therapy studies.

By monitoring bone marrow-derived pCHSPCs in a humanized mouse model, their role in increasing the tumor size in both a flank C32 melanoma model and an intracranial glioblastoma model was validated. In addition to having increases in pCHSPCs correlating with increased tumor volume and fold change, the pCHSPCs appeared to be targeted by the antiviral chemotherapeutic agent, Interferon alpha-2b (Intron A), which significantly decreased tumor size following 2 weeks of treatment. The multi-parametric flow cytometry approach permitted isolation of the CHSPC subsets based upon AC133 expression, and only the AC133⁺ pCHSPC subset possessed pro-angiogenic activity in promoting angiogenesis and human tumor growth in an immunodeficient mouse explant model system.

The ability to track known cellular promoters of angiogenesis in two different murine orthotopic cancer models was demonstrated. By using models that are close to naturally occurring cancers, a clearer grasp of how tumorigenesis and the factors that are necessary for both promoting and disrupting tumor growth can be accurately studied. The pCHSPCs transplanted into a murine xenograft model were shown to mobilize in response to tumor growth, and were decreased upon treatment with a chemotherapeutic agent.

### Example 2

In this Example, patients having sickle cell disease with vaso-occlusive crisis were evaluated for pCHSPC:aCHSPC ratios.

Twenty-four pediatric patients (age<18 years of age) who were inpatient with sickle cell disease with a vaso-occlusive event were used in this Example. Six-eight milliliters of peripheral blood were collected from each patient and analyzed for pCHSPC:aCHSPC ratios as described in Example 1.

The pCHSPC:aCHSPC ratio for patients with sickle cell disease with vaso-occlusive events ranged from 0.24 to 1.0 (average of all patients was 0.73). Interestingly, some patients exhibited some recovery occurring with prolonged events which lead to the ratio increasing into the healthy range or even the active angiogenesis range (average was 2.05; range was 1.24-3.83). Recovery of the pCHSPC:aCHSPC ratio into the healthy range or active angiogenesis range could be an indication of the active remodeling occurring because of the occlusion.

These results demonstrate that the pCHSPC:aCHSPC ratio can be used to determine vasculopathy as a result of vascular inflammation. Inflammation is a key trait of sickle cell disease, which is also a known cause of vascular disease. These results also indicate that pCHSPC appear to be released from the bone marrow to participate in vascular remodeling following a vaso-occlusive event. Prolonged inflammation could also lead to the exhaustion of pCHSPC populations as the cells are released to try to mediate vessel remodeling.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method for detecting a pro-angiogenic circulating hematopoietic stem and progenitor cells (pCHSPC) and an anti-angiogenic circulating hematopoietic stem and progenitor cells (aCHSPC) in a sample obtained from a subject, the method comprising:
obtaining a sample from a subject, wherein the sample is selected from the group consisting of peripheral blood and bone marrow;
detecting a pCHSPC by contacting the sample with an antibody that specifically binds a cell surface marker selected from the group consisting of CD45^{dim}, CD34, CD31, AC133, CD14, CD16, CD41a, CXCR4, LIVE/DEAD, CD38, and combinations thereof, and detecting the resultant binding by multi-parametric flow cytometry; and
detecting an aCHSPC by contacting the sample with an antibody that specifically binds a cell surface marker selected from the group consisting of CD45^{dim}, CD34, CD31, AC133, CD14, CD16, CD41a, CXCR4, LIVE/DEAD, CD38, and combinations thereof, and detecting the resultant binding by multi-parametric flow cytometry.

2. The method of claim 1 further comprising calculating a ratio of pCHSPC to aCHSPC in the sample by determining a number of pCHSPC detected by multi-parametric flow cytometry and by determining a number of aCHSP detected by multi-parametric flow cytometry.

3. The method of claim 1, wherein the subject has or is suspected of having cancer.

4. The method of claim 3, wherein the cancer is selected from the group consisting of melanoma and glioblastoma.

5. The method of claim 1, wherein the subject is undergoing anti-cancer treatment.

6. The method of claim 5, wherein the anti-cancer treatment is interferon treatment.

7. A method of monitoring the efficacy of an anti-cancer treatment, the method comprising:
obtaining a peripheral blood sample from a subject prior to undergoing anti-cancer treatment;
determining a pre-treatment ratio of pro-angiogenic circulating hematopoietic stem and progenitor cells (pCHSPC) to anti-angiogenic circulating hematopoietic stem and progenitor cells (aCHSPC) for the peripheral blood sample;
administering the anti-cancer treatment;
obtaining a post-treatment peripheral blood sample from the subject; and
determining a post-treatment ratio of pCHSPC to aCHSPC for the post-treatment blood sample, wherein a decrease in post-treatment ratio of pCHSPC to aCHSPC as compared to the pre-treatment ratio of pCHSPC to aCHSPC indicates effectiveness of the treatment.

8. The method of claim 7, wherein the anti-cancer treatment is selected from the group consisting of chemotherapy, antibody therapy, radiation therapy, and combinations thereof.

9. The method of claim 7, wherein the anti-cancer treatment is administered for a period of about 21 days.

10. A method for reducing symptoms of a disease, disorder or condition associated with active angiogenesis in a subject, the method comprising administering anti-angiogenic circulating hematopoietic stem and progenitor cells (aCHSPC) to the subject.

11. The method of claim 10, wherein the subject has or is suspected of having cancer.

12. The method of claim 11, wherein the cancer is selected from the group consisting of melanoma and glioblastoma.

13. The method of claim 10, wherein the subject is undergoing anti-cancer treatment.

14. The method of claim 13, wherein the anti-cancer treatment is interferon treatment.

15. The method of claim 10, wherein the subject is pregnant.

16. The method of claim 10, wherein the disease, disorder or condition associated with active angiogenesis is selected from the group consisting of cardiovascular disease, cerebrovascular complications and combinations thereof.

17. The method of claim 10 further comprising administering pro-angiogenic circulating hematopoietic stem and progenitor cells (pCHSPC) to the subject.

18. The method of claim 17, wherein, upon administration, the subject has a ratio of pCHSPC to aCHSPC of from about 1.2 to about 1.8.
